(19) United States
(12) Patent Application Publication
(10) Pub. No.: US 2011/0100921 A1
(43) Pub. Date: May 5, 2011

(54) Device for Separating Particles in and from Liquids and Use of Said Device in Biotechnology, Biological Research, Diagnostics and the Treatment of Diseases

(76) Inventor: Hans-Werner Heinrich, Greifswald (DE)

(21) Appl. No.: 12/993,196

(22) PCT Filed: May 12, 2009
(86) PCT No.: PCT/DE2009/000673

§ 371 Date: Dec. 29, 2010

(30) Foreign Application Priority Data
May 17, 2008 (DE) 10 2008 024 106.7

(51) Int. Cl. A61M 1/36; B01D 15/08; A61M 1/00

(52) U.S. Cl. 210/670; 210/289; 210/143; 210/691

(57) ABSTRACT

The separation of particles in or from liquids is a necessary method in many procedures in biotechnology, biological research, diagnostic, and treatment of diseases. Commonly, methods are used which are based on differences in density (sedimentation) or size (filtration) of the particles.

The invention discloses a device which can be used to concentrate particles within a cylinder by means of a free movable positioning of at least one separating membrane and one plunger within the cylinder. The device is suitable for the time unlimited use in a closed circuit. The invention further discloses a method for the preferable continuous separation of particles.

The invention describes a device for separation of particles from liquids, comprising a cylinder (1) in which is movable situated at least one filter-disc (2), which is solidly mounted to a filter piston (3), and a plunger-disc (4), so that by moving the filter piston (3) the filter-disc (2) and the plunger-disc (4) can be moved together, and the filter-disc (2) separately through the cylinder. The cylinder (1) is closed at the end facing the filter-disc and comprises at least one inlet valve (5) and one outlet valve (5), and further gaskets which seal filter-disc and plunger-disc.

The invention describes a device which can be used to enrich particles from liquids by using a freely mobile assembly of at least one separation membrane and one plunger/piston within a cylinder. The device can be used for an unlimited time in a closed circuit. In addition, the description we will reveal a special form in the shape of a syringe in which the samples within can be analyzed.

The separation of particles in or from liquids is a necessary process step for many procedures in biotechnology, biological research, diagnostics, and treatment of diseases. Typically methods are used which are based on particle differences in density (sedimentation), or size (filtration). Specific “recognition molecules” (ligands) like antibodies, peptides, or nucleic acids can be used as auxiliary support for the separation of biological particles, which can be identified by specific surface structures, and in combination with further methods like flow cytometry or magnetic separation procedures.

Particle separation within the closed compartment is of practical interest for many problems, e.g. maintenance of sterility, protection from infection, and for the continuous separation for biotechnological or medical usage.

Therefore, it is the assignment of the invention to disclose a simple and efficient device for the separation of particles in and from liquids and its use, as well as a simple and efficient procedure for preferable continuous separation from particles from liquids.

The assignment is solved according the claims.

The invention also reveals a new combination of pumping and filtration. The particles to be separated can be e.g. virus, bacteria, protozoa, somatic cells different sizes (e.g. fat cells), magnetizable or other particles of different substances, which are combined with specific ligands on their surface.

### DESCRIPTION OF THE INVENTION

The device comprises one cylinder with inlet and outlet valves according to the intended use, and at least one filter disc, which can be moved independently from the piston. The piston has the function of suction and/or pressing out of the specimens. The filter disc is solid connected with the filter plunger. The filter plunger is separately located inside of the piston plunger. The filter disc is positioned towards the liquid. A special form with only one piston is also revealed.

FIG. 2 illustrates the principle of design and function. FIG. 1 shows one especially preferred execution of the device.

The invention applies especially to a device to separate particles from liquids with or comprised of:

A cylinder (1) within at least one filter disc (2) is located, solid connected with the filter-plunger (3), and one slidable supported piston-disc (4), so that by means of the filter-plunger (3) at least one filter-disc (2) and the piston-disc (4) can be moved together, and the filter-disc (2) separately through the cylinder (1), and the cylinder (1) which is closed at the end facing at least one filter-disc and provided with at least one inlet and outlet valve (5);

Gaskets (6) which are fitted with at least the filter-disc (2) and the piston-disc (4), in which the gaskets (6) are preferable gasket rings and cover preferable the complete cylinder wall.

If necessary further valves and tubing.

Especially useful is the configuration when the cylinder (1) is prepared as a hollow cylinder to which one inlet and outlet valve (5) is attached at the cylinder bottom, and in with at least one filter-disc (2) is located above the cylinder base, and the piston-disc (4) behind the filter-disc in a manner that filter-disc (2) and piston-disc (4) with the attached gaskets (6) in each case fills the complete diameter of the hollow cylinder, and which are preferable arranged parallel to the cylinder bottom, and the piston-disc (4) has a hole preferable in central location, especially a drill hole, through which the filter-piston (3) can be moved, preferably coaxial to the cylinder (1). To prevent the non-wanted transfer of matter through the openings, they are preferably provided with gaskets, especially as washer through which the filter-piston (3) is directed.

It is especially favorable when the piston-disc (4) is solidly connected to the piston plunger (7), so that the piston-disc (4) by means of the piston-plunger (7) can be separately moved within the cylinder (1).

A beneficial advancement of the invention is the formation of the piston plunger (7) as a pipe and/or is situated respective moved coaxial to the cylinder (1).

Especially favored is a device where the filter-plunger (3) is located at least partially within the piston plunger (7) so it respectively can be moved inside.

Favored is the implementation where the piston plunger (7) in its inner site the filter-plunger (3) fixates, and so allows the free sliding of the filter plunger (3) coaxial to the cylinder (1).

Another advancement of the invention is that the piston-disc (4) is freely movable mounted to/on the filter-disc (3), so that the piston-disc (4) for example can be freely moved together with the filter-plunger (3) in the opposite direction of the closed cylinder site; can remain there, and/or can be separately shifted in direction of the closed end of the cylinder.

Of advantage for the use, especially for the continuous function according to the invention, is when on the filter-plunger (3) on any place at least one arrest device is located, which holds and fixes the piston-disc (4) by moving of the filter-plunger (3) in the direction of the inlet-outlet valve (5). The arresting can be executed in only one or both directions.

A further development according to the invention is that on the piston-disc (4) at least one mechanism/device is provided which arrests the piston-disc (4) by moving the filter-plunger (3) in direction inlet-outlet valve (5). The arrest can be performed in only one or both directions.

For different applications, especially for the extra-corporeal removal of substances and/or particles from blood, it is favorable to attach a filter in front of the inlet-outlet valve (5), which has a preferable mesh size which is smaller than the diameter of the particles, especially capturing micro-particles which are located within the cylinder (1).

Another further development is that the device is equipped with at least one inlet valve (5) and one outlet valve (5).

Of advantage for the use, especially for the extra-corporeal removal of substances and/or cells, is that at least one outlet-valve (5) is covered with one filter, preferable with a mesh size of at least one of the filter-discs (2).

An especially useful embodiment of the device is the presence of several filter-discs (2) of different mesh sizes, which are separately fixed to filter-plungers (3), and which are situated in front of the plunger-disc (4).

In another useful embodiment, the pore size of at least one filter disc (2) can vary from 0.01 to 1,000 μm.

A further embodiment of the device is the shape of a syringe, preferably within the typical measurements of a syringe; respectively the sub-units of the device have the size and the shape of the syringe units or are adapted to them, and/or are applicable to handle specimens in a syringe body.

Especially for the continuous use, the device consists of at least but preferably two appliances according claims 1-16, allowing a continuous and time-independent application by connecting the devices by means of tubes and valves in a way that one device is in use whereas the other device can be regenerated.

In a further development of the invention, the device is designed for its use in a circuit, preferably integrated in the circuit.

Another aspect of the invention applies to the use of the device according to the invention, in which the cylinder (1) is filled with preferably a liquid due to simultaneous moving of the filter-disc (2) and the plunger-disc (4) and the open inlet-valve (5).

It is especially of advantage when the cylinder compartment (1) between the filter-disc (2) and the closed end of the cylinder is passively filled by using appropriate valves, preferable the inlet valve (5).

A further embodiment is use of the device, so, that with closed valves, especially closed valve (5), the filter-disc (2) is pushed through the liquid by means of the filter-plunger (3), whereas the plunger-disc (4) maintains in the position “cylinder filled”.

In another favored use, particles, especially micro-particles, are located between the filter-disc (2) and the closed end of the cylinder which have a greater size than the pore size of the filter.

For different embodiments it is of advantage when several filter-discs (2) with different pore sizes are used which are separately connected with filter-plungers (3) and positioned in front of the plunger-disc.

Approved is a device, especially for the enrichment of particles from liquids with a diameter of >0.01 μm, preferably between 0.1 and 1,000 μm.

For the application of the device preferably liquids of any chemically composition can be used, which are applicable for keeping the enriched particle in a flexible state.

Especially useful is the device for the purification of liquid suspensions of biotechnological processes, samples from the environment, wash samples from body cavities, blood and/or se- and excretes.

The device is especially helpful for its use to separate particles: viruses, bacteria, protozoa, cell organelles, inclusion bodies, somatic cells and/or synthetic particles, whereas the synthetic particles are the result of a biotechnological process or synthetic biopolymers.

Especially suitable is the use of biopolymer particle which have paramagnetic characteristics and/or are equipped with specific ligands.

Of advantage is the device especially for its extra-corporeal use within a circuit respectively integrated in an extra-corporeal circuit.

A further embodiment, especially for the continuous application, comprises of at least but preferably two of the devices combined by tubes and valves in a way which allows the continuous use of the device, meaning that one device is in use whereas the second gets regenerated.

Of particular advantage is the use of the device for the extra-corporeal blood treatment and/or the extra-corporeal diagnostic or therapeutic application.

A further embodiment, especially for the medical-technical use, is the device in the form of a syringe allowing the treatment of the sample within the syringe body.

A further aspect of the invention describes a procedure for the preferable continuous separation of particles from liquids, whereby a hollow cylinder and a cylinder base forming the cylinder (1), whereby that in the area of the cylinder base an inlet-outlet valve (5) is situated, a particle containing liquid is filled in the cylinder, whereby that by the simultaneous movement of filter-disc (2) located above the cylinder base, and a plunger-disc (4) which rests on the filter-disc (2) the liquid is drawn within the cylinder, whereby that after closing the inlet-outlet valve (5) and especially arresting the plunger-disc (4) the filter-disc (2) is pushed through the liquid in the direction of the cylinder basis, whereby that all particles with a diameter larger than the pores (mesh size) of the filter-disc (2) will be pushed in front of the filter-disc (2) and concentrated in the room between filter-disc and cylinder basis, whereby that the particles by means of a valve (5) can be removed or transported from the cylinder.

It is of especially advantage when for the treatment the device is used according claims 1-17.

An especially favorable embodiment of the procedure is that the filter-disc (2) is solidly connected with the filter-piston (3), and the plunger-disc (4) rests as flexible disc on the filter-disc (2) facing away from the sample, whereby by the open valve (5) the filter-disc (2) is moved away from the valve (5), whereby the plunger-disc (4) is passively carried along with the filter-disc, whereby the liquid is drawn within the cylinder. For the separation procedure the inlet valve (5) is closed, and by pushing the filter-piston (3) the filter-disc (2) is moved through the liquid, whereas the plunger-disc (4) remains in the upper position.

The procedure according to the invention is especially favorable when it is performed according the claims 18-38 of the invention.

In particular, the procedure according the invention is very suitable for the extra-corporeal removing of substances and/or particles from blood, whereas the blood after proper coagulation is transported by means of a blood pump into the device according claim 10-17, especially with the features according 10, 11 and/or 13, whereas the filter-disc (2) and the plunger-disc (4) are located at the lower end of the cylinder and the cylinder contains micro-particles which preferably bind molecules, particles including disease causing agents and/or somatic cells, and whereas after filling the cylinder compartment with blood all valves (5) are closed and by putting pressure on the filter-piston (3) the filter-disc (2) is moved through the blood, whereas the micro-particles and the molecules, particles including disease causing agents and/or somatic cell bound to them, are concentrated in the space between the filter-disc (2) and the basis of the cylinder, and whereas the remaining blood in the cylinder located between the filter-disc (2) and the plunger-disc (4) can be transferred back to the organism through a outlet valve, preferably located above the filter-disc (2) by pushing the Plunger-disc (4) in the direction of the end of the cylinder.

It is favorable, especially for the continuous use, to regenerate the target-loaded micro-particles. If anti-bodies are used as specific ligands, regeneration will be preferably done by lowering the pH to <3.0.

Other preferable characteristics and embodiments of the invention are elucidated in the following description which is not complete and can be easily adopted to related procedures by a specialist.

By a simultaneous movement of plunger-disc (4) and filter-disc (2), the specimen is drawn into the cylinder by passing the valve (5). The filling of the cylinder is also possible when the plunger-filter-discs are in distal position by means of a valve system.

When all openings of the common valve systems are closed, the filter-disc can be pushed through the liquid. All particles with a bigger size than the pore size of the filter will be concentrated in front of the filter in direction of the proximal cylinder end. Here, they can be removed by the use of a valve, and according the need discarded, further processed, and/or replaced by new particles. The FIG. 2 demonstrates the principle of filling, concentration and separation of particles, emptying, further processing of the particles and/or replacing by new particles. It is easy for a specialist to adopt the principle to other technological necessities by means of additional valves, filter, pumps, gaskets, etc. and in combination with the disclosed invention.

The leak-tightness of the system is secured by common procedures, materials and devices.

A special form of the device is illustrated in the FIGS. 3A and 3B. The filter is solid connected to the filter piston/axis. The plunger-disc (4) is located as free movable disc on the filter-disc, facing away from the sample. The valve is open, and the filter is moved away from the valve. The plunger-disc is passively moved by the filter-disc and draws the liquid into the cylinder. The in-let valves have to be closed for the separation/concentration. The filter will be pushed through the liquid by pressing the piston. The plunger-disc remains in the upper (distal) position. Certain mechanisms are located on the filter-piston which arrests the plunger-disc. Their location is arbitrary. The arrest point defines how far the filter-disc can be moved through the liquid during the valve-closed situation. The outlet-valve has to be opened to remove the particles in front of the filter-disc. By pressing on the filter-piston the arrested plunger-disc pushes the liquid in the direction of the outlet, till the filter-disc reaches the cylinder end or another mechanical holdup.

Another embodiment is distinguished by one filter, located in front of the outlet, which retains the wanted particle fraction. That allows the multiple repetitions of the described filling, and/or separation/concentration, and emptying procedure. The preferred particles can be used several times with the same or different liquids. The described particles can so remain in the device and serve as solid support for common diagnostic procedures, e.g. enzyme immune reaction (Enzyme Immune Assay), or they can be removed from the device by a different outlet.

The FIG. 4 illustrates the principal procedure of filling, separating/concentrating, and emptying, and analyzing.

The device can be used in a closed circuit. At least two devices are necessary for the continuous and time-wise unlimited use, which are alternatively operated. Thereby, during ongoing operating of the devices, particles can be removed in front of the filter-disc and/or replaced by a new one.

One application of the device is its use in a closed circuit within an extra-corporeal blood circuit for the purpose of removal of substances and/or particles from blood (FIG. 5). Purpose of the usage is the recognition of pathologic conditions and their treatment. The device according to the invention is especially useful for the continuous removal of large amounts of cells from the blood flow, as it is e.g. necessary for certain tumors, infections, or systemic inflammations. By means of a blood pump, the anti-coagulated blood is pumped in the device. Filter-disc and plunger-disc are in distal/lower position. Particles with the requested/desired function are located in the cylinder. They bind e.g. molecules, particles including pathogens and/or somatic cells. The functionalized particles are kept in suspension by appropriate technological solutions, like interior design of the cylinder, vibration, stirring, shaking, etc. The device can be temperate by common procedures if necessary. When the particles are loaded with the targets, the blood is guided via the valve in the other device, which contains the functionalized particles.

All valves will be closed for the removal of the loaded particles. By pressing the filter piston, the filter will be moved through the blood. The particles will be concentrated in the space between the filter and the proximal end of the cylinder. The used particles can be substituted easily by means of common valve systems. The regeneration of the used particles is possible. The lowering of the pH <3.0 has been proven practical when antibodies are used as specific ligands. But also other procedures, e.g. enzymatic or chemical cleavage of pre-formed sites can be used, especially for the liberation of living somatic cells from the particles. The device and the disclosed procedure allow a cost-effective use of the expensive ligands (e.g. monoclonal antibodies) in especially for disease treatment, due to their multiple recycling.

At the end of the extra-corporeal treatment, the remaining blood will be directed back into the organism by moving the plunger disc in direction of the end of the cylinder.

The procedure is suitable for the separation of cells of different size (e.g. fat cells), as also functionalized particles, which are added to the liquid for the purpose to separate and isolate specific targets. Substance and characteristics of the used particles are defined by the targets. Particles with a big surface will be used preferably for the binding of molecules. For the separation and isolation of particles or cells, bio-polymers without a big internal surface will be used preferably.

Whereas the regeneration of immune adsorbers for the purpose of molecules is state of the art, the device according to the invention makes it for the first time possible to regenerate and reuse solid support, preferable particles, for the removal of specific cells from the blood flow.

Particles of different fictionalization can be used especially for the therapeutic application as within extra-corporeal circuit, e.g. for the removal of proteins like complement factors or interleukins in combination particles which target specific cells. The appropriate combination depends on the pathogenic function of the targets.

The use of paramagnetic particles is also possible. After being loaded with the targets and being removed from the device, they can be treated or processed by the common use of magnetic fields.

The pore size of the filters depends on the nature of the liquid and the size of the particles. Especially applicable for blood are pores with a diameter ≧20 μm. The functionalized particles have to be bigger than size of the pores to facilitate their separation within the device.

The simultaneous use of several filter-discs within one device is also possible. Separate inlet/outlet valves support the separate removal of the particular particle fraction.

### LEGEND TO THE FIGURES

Principle configuration:
Cylinder (1)
Filter-disc (2)
Filter-piston (3)
Plunger-disc (4)
Inlet and outlet valve (5)
Caskets (6)
Plunger-piston (7)
Additional valves and tubes by necessity

The numeration of the FIGS. 1-3 is accordingly applicable for the other figures.

FIG. 1: Principle of separation

FIG. 2: Two-plunger device, demonstration of the principle of filling, particle separation, and emptying and/or processing

FIG. 3: One-plunger variation, as special form of the device
A: Plunger disc arrest in both directions
B: Plunger-disc arrest in one direction

FIG. 4: Function of the one-plunger variation

FIG. 5: Device as part of an extra-corporeal circuit

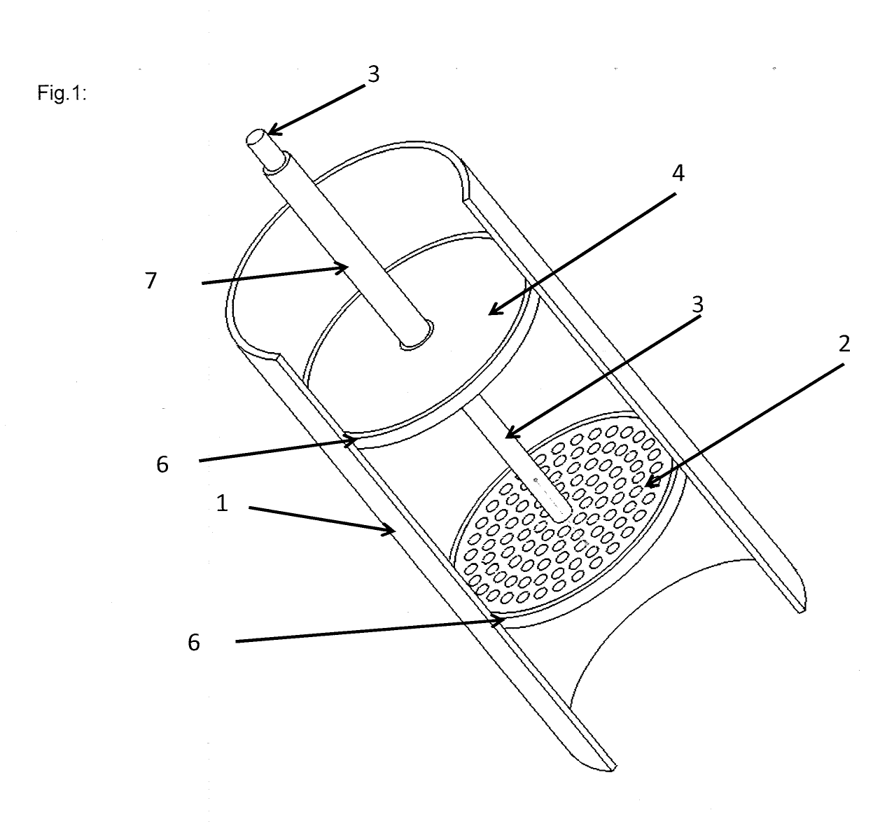

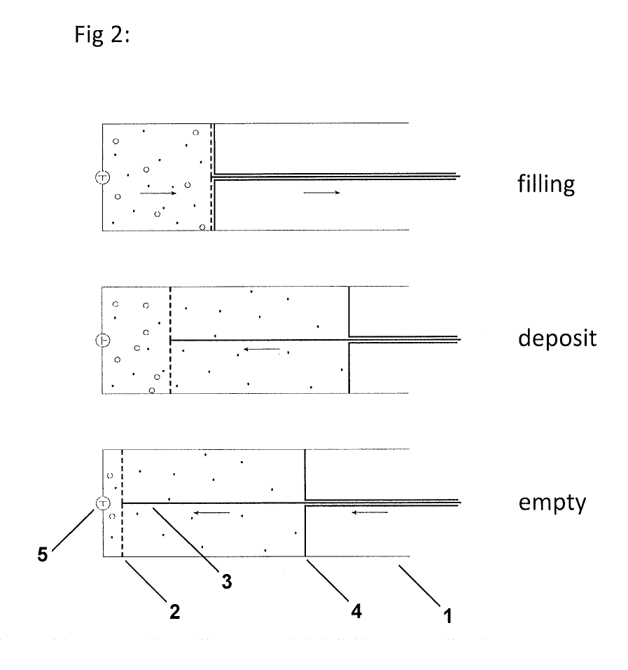

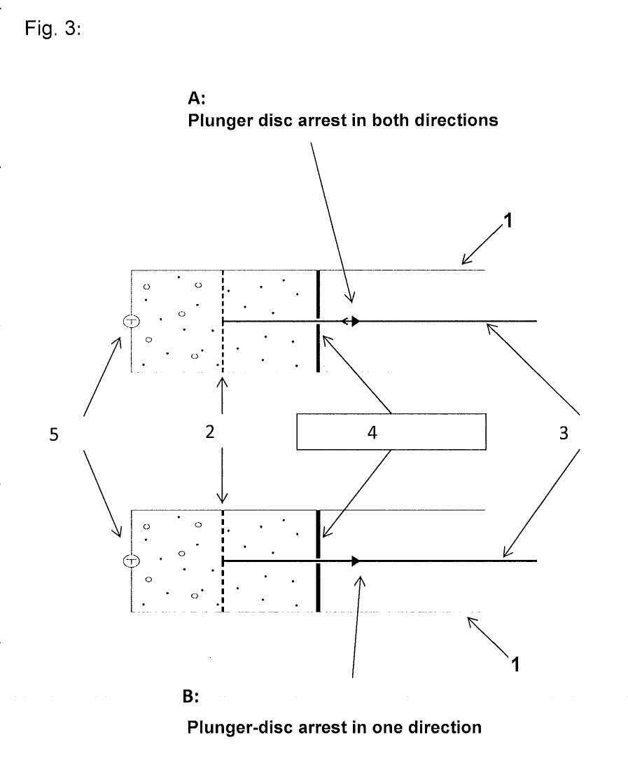

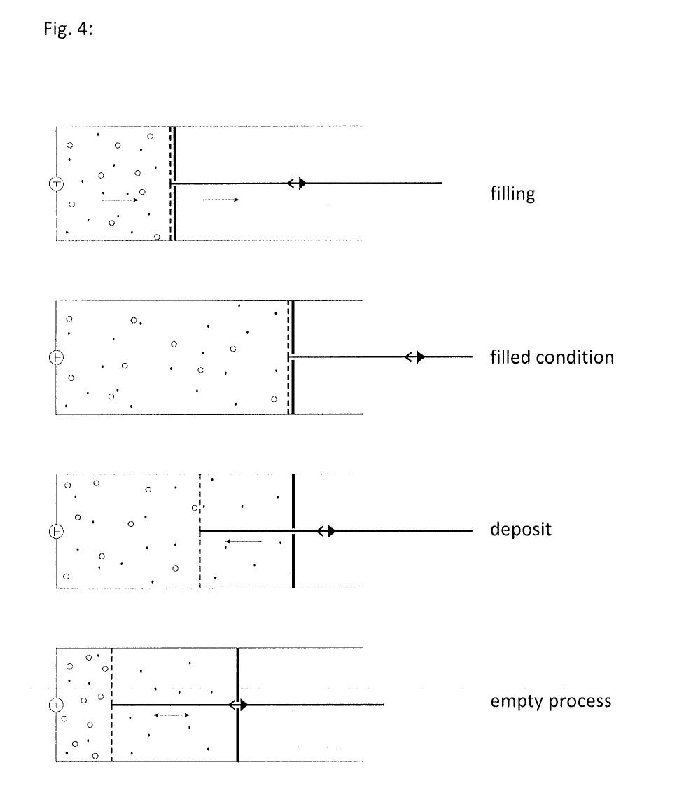

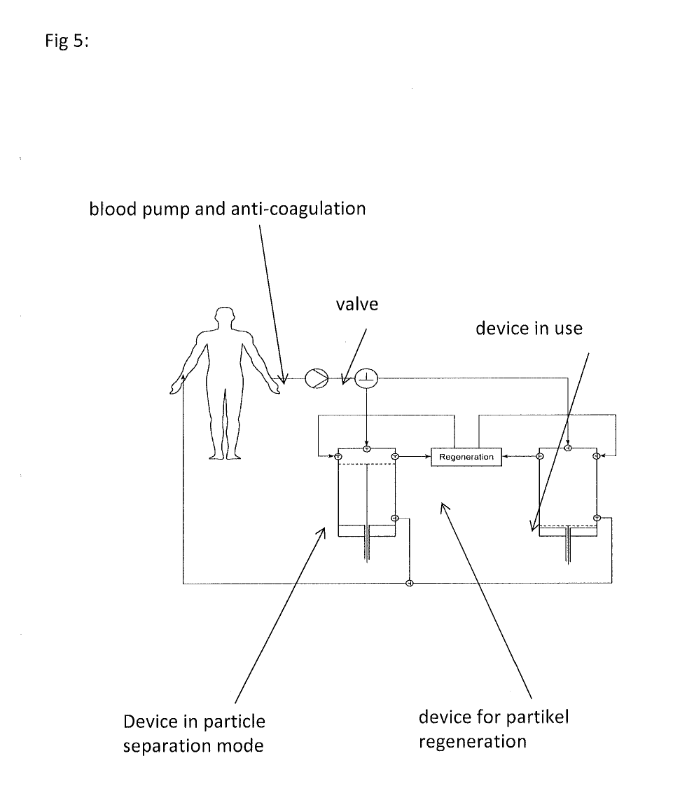

All stated characteristics in the description of the invention, the claims, and the figures can be used separately or in any combination for the realization of the invention in their different embodiments.

1. A device for the removal of particles from consisting of:
a. A cylinder (1) containing at least one filter-disc (2), which is solidly connected with a filter piston (3), and a plunger-disc (4), which is movable mounted, so that by means of the filter piston (3) at least one filter-disc (2) and the plunger disc (4) can be moved together, and the filter-disc (2) separately through the cylinder (1); the cylinder (1) is closed at the end which faces the filter-disc (2) and comprises at least one inlet valve and one outlet valve (5), whereas the outlet valve comprises a filter with pore sizes smaller than the size of functionalized particles situated between the filter-disc (2) and the end of the cylinder (1), whereas the pores of the filters have a diameter ≧20 μm;
b. Functionalized particles between the filter-disc (2) and the end of the cylinder (1) with a size larger than the pore size of the filters
c. Means for sealing (6) of at least the filter-disc (2) and the plunger-disc (4), whereas the sealing (6) is preferably a gasket ring touching the cylinder wall over the complete cross section.
d. Additional means of valves and tubes if necessary.

2. Device according to claim 1, wherein the cylinder (1) is formed as a hollow cylinder with one cylinder basis in which at least one inlet-outlet valve (5) is positioned, and comprises at least one filter-disc (2) located above the cylinder basis and plunger-disc (4) located above the cylinder basis and the at least one filter-disc (2), whereas the filter-disc (2) and the plunger-disc (4) equipped with gaskets (6) fill the cross section of the cylinder, and are preferably positioned parallel to the cylinder basis, wherein the plunger-disc (4) comprise an opening especially a central boring wherein the filter-piston (3) can be moved preferably coaxial to the cylinder (1).

3. Device as claimed in one of the claims 1-2, wherein the plunger-disc (4) is firmly mounted to the plunger piston (7), in such a way that the plunger-disc (4) can by moved separately through the cylinder (1) by means of the plunger piston (7).

4. Device as claimed in claim 3, wherein the plunger piston (7) is a hollow pipe positioned said guided coaxial to the cylinder (1)

5. Device as claimed in one of the claims 3-4, wherein the filter piston (3) is at least partially situated said guided within the plunger piston (7)

6. Device as claimed in one of the claims 3-5, wherein the plunger piston (7) is positioned free movable inside the filter piston(3) preferably coaxial to the cylinder (1)

7. Device as claimed in one of the claims 1-6, wherein the plunger-disc (4) is flexibly placed on the filter piston (3).

8. Device as claimed in one of the claims 1-7, comprising at least one arresting mechanism on the filter piston (3), which can be located in any distance to the filter-disc (2), and therefore catches the plunger-disc (4) by moving the filter piston (3) in the direction inlet/outlet valve (5) in a certain position, at which the arrest can be carried out in only one or both direction of filter piston movement.

9. Device as claimed in one of the claims 1-8, comprising at least one mechanism on the plunger-disc (4), which arrests the plunger-disc (4) by moving the filter piston (3) in direction inlet/outlet valve (5) in a distance free of choice, whereat the arrest can be achieved in only one or both direction of filter piston movement.

10. Device as claimed in one of the claims 1-9 9, wherein the inlet and/or outlet valve (5) comprises a filter with a pore size smaller than the size of the particles, especially micro-particles, which are located inside the cylinder (1)

11. Device as claimed in one of the claims 1-10, comprising at least one inlet valve (5) and at least one outlet valve (5).

12. Device as claimed in claim 11, comprising at least one outlet valve (5), with a filter preferably with the pore size greater or analogous to the filter-disc (2).

13. Device as claimed in one of the claims 1-12 further comprises several filter-discs (2) with different pore sizes located in front of the plunger-disc (4), each with mounted on separate filter pistons (3).

14. Device as claimed in one of the claims 1-13, wherein the at least one filter-disc (2) is characterized by pore sizes between 0.01 and 1,000 μm.

15. Device as claimed one of the claims 1-14, wherein it has the shape of a syringe and/or are applicable to handle specimens in a syringe body.

16. Device as claimed in one of the claims 1-15 comprising at least two embodiments of the device, so that preferably a time unlimited usage is feasible, wherein by means of tubes and valves one device is always in operation whereas the other is regenerated.

17. Device as claimed in one of the claims 1-16, wherein the device is formed for the use in a circular flow, preferably integrated in a circular flow.

18. Use of the device as claimed in one of the claims 1-17, wherein the cylinder (1) is filled by simultaneously moving of the filter-disc (2) and the plunger-disc (4) while admission valve is opened.

19. Use of the device as claimed in claim 18, wherein the hollow cylinder between the filter-disc (2) and the closed basis of the cylinder is filled passiv via adequate valves, preferable valve (5)

20. Use of the device as claimed in one of the claims 18-19, wherein at closed valves, preferably closed valve (5) the filter-discs by means of the filter pistons (3) are pushed through the liquid, whereas the plunger-disc (4) remains in the position “filled cylinder”

21. Use of the device as claimed in one of the claims 18-20, wherein the particles between the filter-disc (2) and the cylinder basis and/or between the filter-discs have a diameter greater than the pore size of the filter-disc.

22. Use of the device as claimed in on of the claims 18-21, wherein multiple filter-disc (2) with different pore diameter, connected with separate filter pistons (3) were used and which are operated in front of the plunger-disc (4).

23. Use of the device as claimed in one of the claims 18-22 for the enrichment of particles from liquids with a diameter of >0.01 μm, preferably between 1 and 1,000 μm.

24. Use of the device as claimed in one of the claims 18-23, wherein the liquids exhibit any chemically composition, which is applicable for keeping the enriched particle in a flexible state.

25. Use of the device as claimed in one of the claims 18-24, wherein the liquids are suspensions of biotechnological processes, samples from the environment, wash samples from body cavities, blood and/or se- and excretes.

26. Use of the device as claimed in one of the claims 18-25, wherein the particles can be aggregates like viruses, bacteria, protozoa, cell organelles, inclusion bodies, somatic cells and/or synthetic particles

27. Use of the device as claimed in one of the claims 18-26, wherein the synthetic particles are the result of a biotechnological process or synthetic biopolymers.

28. Use of the device as claimed in one of the claims 18-27, wherein the synthetic particles may have paramagnetic characteristics and/or are equipped with specific ligands

29. Use of the device as claimed in one of the claims 18-28, wherein the device can be integrated in a circuit.

30. Use of the device as claimed in one of the claims 18-29, preferably a device as claimed in claim 13, wherein at least two embodiments combined by tubes and valves in a way which allows the continuous preferably a time-unlimited use of the device, meaning that one device is in use whereas the second gets regenerated.

31. Use of the device as claimed in one of the claims 18-30 for the extra-corporeal blood treatment and/or the extra-corporeal diagnostic or therapeutic application.

32. Use of the device as claimed in one of the claims 18-31, preferably a device as claimed in claim 12, wherein the application is carried out in the form of a syringe and/or allowing the treatment of the sample within the syringe body.

33. Method for the preferable continuous separation of particles from liquids, wherein a hollow cylinder and a cylinder base forming the cylinder (1), whereby that in the area of the cylinder base an inlet-outlet valve (5) is situated, a particle containing liquid is filled in the cylinder, whereby that by the simultaneous movement of filter-disc (2) located above the cylinder base, and a plunger-disc (4) which rests on the filter-disc (2) the liquid is drawn within the cylinder, whereby that after closing the inlet-outlet valve (5) and especially arresting the plunger-disc (4) the filter-disc (2) is pushed through the liquid in the direction of the cylinder basis, whereby that all particles with a diameter larger than the pores (mesh size) of the filter-disc (2) will be pushed in front of the filter-disc (2) from where they can be removed or transported from the cylinder via a valve, preferably valve (5).

34. Method as claimed in claim 33, wherein a device according to claims 1-17 is used.

35. Method as claimed in one of the claims 32-33, wherein the filter-disc (2) is solidly connected with the filter-piston (3), and the plunger-disc (4) rests as flexible disc on the filter-disc (2) facing away from the sample, whereby by the open valve (5) the filter-disc (2) is moved away from the valve (5), whereby the plunger-disc (4) is passively carried along with the filter-disc, whereby the liquid is drawn within the cylinder (1) and for the separation procedure the inlet valve (5) is closed, and by pushing the filter-piston (3) the filter-disc (2) is moved through the liquid, whereas the plunger-disc (4) remains in the upper position.

36. Method as claimed in one of the claims 33-35, wherein it is performed according the claims 18-32 of the invention.

37. Method as claimed in one of the claims 33-36 for the extra-corporeal removing of substances and/or particles from blood, whereas the blood after proper coagulation is transported by means of a blood pump into the device according claim 10-17, wherein the filter-disc (2) and the plunger-disc (4) are located at the lower end of the cylinder (1) and the cylinder (1) contains at the lower end micro-particles which preferably bind molecules, particles including disease causing agents and/or somatic cells, and whereas after filling the cylinder compartment with blood all valves (5) are closed and by putting pressure on the filter-piston (3) the filter-disc (2) is moved through the blood, whereas the micro-particles and the molecules, particles including disease causing agents and/or somatic cell bound to them, are concentrated in the space between the filter-disc (2) and the basis of the cylinder, and whereas the remaining blood in the cylinder located between the filter-disc (2) and the plunger-disc (4) can be transferred back to the organism through a outlet valve, preferably located above the filter-disc (2) by pushing the Plunger-disc (4) in the direction of the end of the cylinder.

38. Method as claimed in claim 37, wherein the used functionalized particles can be regenerated by use of antibodies as specific ligand preferable by lowering the pH at <3.0.
